(19) United States
(12) Patent Application Publication
(10) Pub. No.: US 2010/0134757 A1
(43) Pub. Date: Jun. 3, 2010

(54) OPTICAL REHABILITATION TREATMENT

(76) Inventor: Sergio ARCONA, Torino (IT)

Correspondence Address: SUGHRUE MION, PLLC, 2100 PENNSYLVANIA AVENUE, N.W., SUITE 800, WASHINGTON, DC, 20037, US

(21) Appl. No.: 12/325,624

(22) Filed: Dec. 1, 2008

(51) Int. Cl. A61B 3/028; A61H 5/00

(52) U.S. Cl. 351/203

(57) ABSTRACT

An optical rehabilitation treatment comprising the steps of:
a) subjecting a patient to an anamnesis of his/her ocular defects;
b) controlling the patient's maximum visual acuity by positioning the patient first at a distance of about 3 meters, and then at a distance of about 1.5 meters, from an octotype;
c) wetting the patient's cornea (2) with a preservatives-free non-reusable physiological saline solution
d) applying to the patient at least one contact lens (400) with an initial optical power and, simultaneously, glasses with at least one ophthalmic lens (300) with an initial optical power;
e) progressively changing said optical means (300, 400) by gradually increasing their relevant optical powers until a visual improvement is obtained;
f) letting the patient with said optical means (300, 400) on for at least one hour, the optical power of each of said optical means (300, 400) being that with which the patient recovered the visual function;
g) removing said optical means (300, 400) after at least one hour has passed;
h) subjecting again the patient to a control of his/her maximum visual acuity, to determine the obtained visual improvement,
   wherein said step d) comprises applying to the patient either at least one diverging contact lens (400) with initial optical power of −5.00 diopters and glasses with at least one converging ophthalmic lens (300) with initial optical power of +5.00 diopters, or at least one converging contact lens (400) with initial optical power of +5.00 diopters and glasses with at least one diverging ophthalmic lens (300) with initial optical power of −5.00 diopters.

### TECHNICAL FIELD

The present invention refers to a rehabilitation treatment for the pathologies of the visual function.

In particular, the present invention refers to a rehabilitation treatment for the cure of maculopathy.

### PRIOR ART

Maculopathy, or macular degeneration, is the main cause of legal blindness among the elderly in Italy and it is broadly widespread in the United States of America too, with about one million sick people.

Two kinds of maculopathy exist: the dry or atrophic kind and the exudative or neovascular or disciformis kind; most of the patients suffer from maculopathy of the first kind.

Dry maculopathy is generally caused by a thinning of the macula's layers, and vision loss typically is gradual; wet maculopathy can result when some blood vessels developed underneath the macula hemorrhage and destroy the macular tissues, thus causing a very rapid and devastating vision loss.

Macular tissues destroyed by either dry or wet maculopathy cannot be repaired.

Up to now, neither the causes of maculopathy nor a definitive cure for it have been certainly identified.

In particular, from the therapeutic point of view, the atrophic form of maculopathy is not susceptible to laser treatment, nor the transplant of the damaged retinal area is easily practicable.

Among the solutions nowadays applied, a patient may use magnifying lenses or specific visual aids (for instance, glasses equipped with Galileian or telescopic lenses systems, or video-magnifiers showing on a monitor, at the desired greatness, the fonts of a text that is made sliding onto a desk, or even computerized or talking devices); an example of a rehabilitation treatment for the cure of maculopathy according to the prior art is represented in FIG. 4, which will be discussed in detail hereinafter.

However, these are palliative and temporary solutions. Therefore, there still subsists the need of identifying a cure method allowing the patient to recover the visual function, at least so as to be self-sufficient.

More precisely, there still subsists the need of identifying a cure method allowing the patient to achieve a permanent visual recover that, moreover, does not require to keep on using visual aids.

### DESCRIPTION OF THE INVENTION

The present invention intends to answer the yet unsatisfied need for a cure method allowing the patient to permanently recover his/her visual function, at least so as to be self-sufficient, without keeping on using any visual aids.

This object is attained thanks to the optical rehabilitation treatment according to the present invention that, advantageously, allows the patient to recover his/her visual function, at least so as to be self-sufficient.

In particular, advantageously, the optical rehabilitation treatment according to the present invention allows the patient to achieve a permanent visual recover that, moreover, does not require to keep on using visual aids.

### SYNTHETIC DESCRIPTION OF THE FIGURES

The present invention will be now described in more detail with reference to the following figures, given by way of non limiting example, in which:

FIG. 1 is a perspective view of a human eyeball;

FIG. 2 is cross section view of FIG. 1;

FIG. 3 is a simplified cross section view of FIG. 1 showing a detail of the macula;

FIG. 4 represents a rehabilitation treatment for the cure of maculopathy according to the prior art; and

FIG. 5 represents the rehabilitation treatment for the cure of maculopathy according to the present invention.

### DESCRIPTION OF A PREFERRED EMBODIMENT

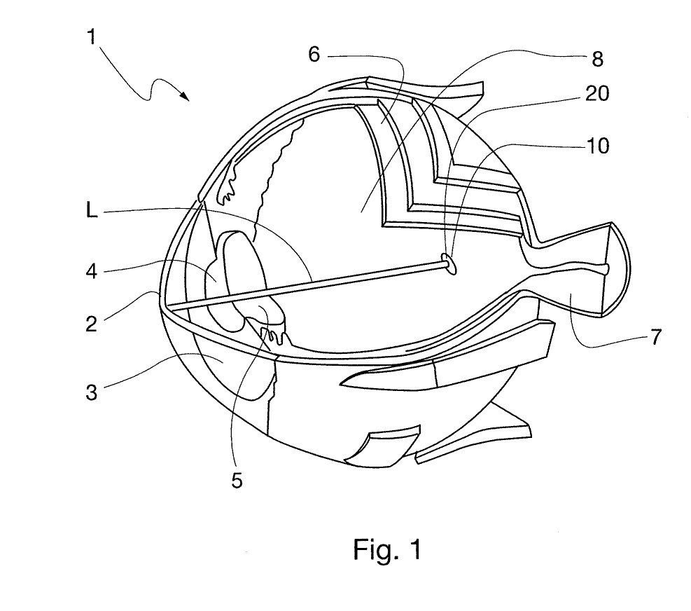

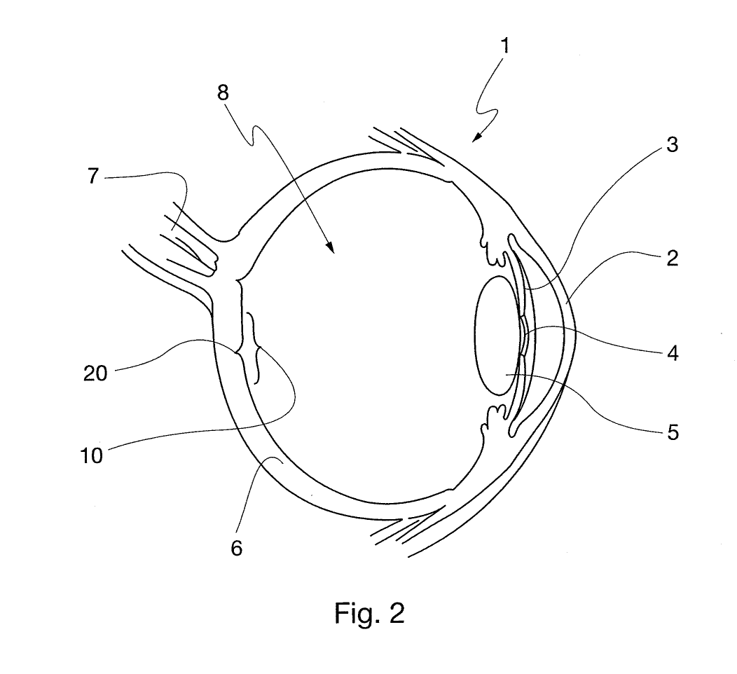

With reference to FIGS. 1 and 2, some of the most important anatomical components of the human eye 1 can be observed; in particular, there are shown the cornea 2, the iris 3, the pupil 4, the crystalline lens 5, the retina 6, the optic nerve 7, the vitreous humor 8, the macula 10 and the fovea 20.

The human eye is the organ which gives humans the sense of sight, allowing them to observe and learn about the surrounding world; the eye allows humans to see and interpret the shapes, colors and dimensions of objects by processing the light they reflect or emit.

Referring to FIG. 1, light waves L from an object enter the eye 1 first through the cornea 2, the clear dome at the front of the eye 1; the light waves L then progress through the pupil 4, the circular opening in the center of the colored iris 3.

Initially, the light waves L are bent or converged first by the cornea 2, and then further by the crystalline lens 5, which is located immediately behind the iris 3 and the pupil 4, to a point located immediately behind the back surface of the lens 5.

The light waves L continue through the vitreous humor 8, the clear gel that makes up about 80% of the eye's volume, and then, ideally, back to a clear focus on the retina 6, behind the vitreous humor 8.

The small central area of the retina 6 is the macula 10, which provides the clearest, most distinct vision of any location in the retina 6: when one looks directly at an object, the light waves L from that object form an image on one's macula 10; the center of the macula 10 is the fovea 20, which is the point of sharpest, most acute visual acuity.

To conclude the process of vision, within the layers of the retina 6, light impulses are changed into electrical signals; then, they are sent through the optic nerve 8 that acts like a cable connecting the eye 1 with the brain, along the visual pathway, to the occipital cortex at the posterior of the brain, where the electrical signals are interpreted or “seen” by the brain as a visual image.

Certain conditions can affect the macula 10 and, in turn, one's central vision; as already said, the most common disease is the maculopathy, or macular degeneration.

The earliest symptoms of maculopathy usually are the images distortion, whereby the objects appear to be deformed and/or smaller than the original size, and the reduction of the ability of fine discrimination, whereby the objects and colors recognition, reading and writing are jeopardized; eventually, a small area of no vision, in the central visual field, can develop and grow in size, and this can progress until people's faces are unrecognizable when looking directly at them; yet, peripheral vision remains unaffected.

Dealing now with the disease specificity, maculopathy consists in the damage of the aforesaid eye's structure named macula 10.

Said macula 10 is located at the centre of the retina 6 and it is useful for recognizing details, while the remainder of the retina 6 is useful for widening the visual field.

More precisely, the retina 6 is a photosensitive membrane, mainly made of layered nerve cells; these cells are differentiated depending on their structure and their function.

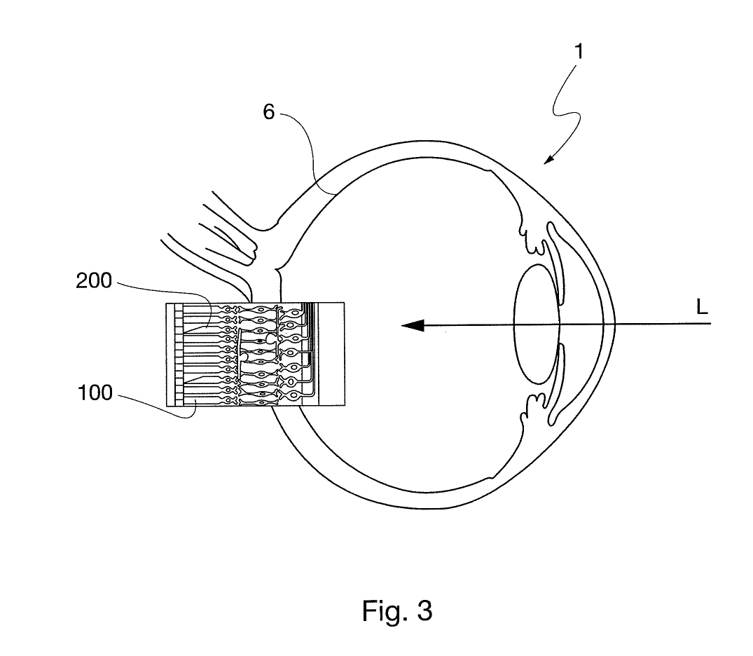

Making now reference to FIG. 3, two types of light-sensitive cells or photoreceptors, the rods 100 and the cones 200, are visible, which are used in image-forming vision; both rods 100 and cones 200 are connected to nerve fibers, so that when these cells are stimulated by light, they send off nerve impulses.

There are about 120 to 130 million rods 100 in each eye, and they are sensitive to dim light, to movement and to shapes; rods 100 cannot detect colors but they are responsible for black and white vision.

The rods 100 are able to assure the twilight view at reduced visual acuity; they are connected to groups of nerve terminations and, therefore, they are able to respond to reduced but diffused stimuli, while they do not have the ability of distinguishing little details of the perceived image.

The highest concentration of rods 100 is in the peripheral retina 6, decreasing in density up to the macula 10; there are no rods 100 at the fovea 20.

There are about 6.5 to 7 million cones 200 in each eye, and they are sensitive to bright light and to colors; cones 200 are responsible for color vision and they require brighter light to function than rods 100 require.

The cones, which are much less sensitive to light than the rods, are sensitive to colors and they permit a high visual acuity, or visus; since they are singly connected to the nerve terminations, they allow to distinguish fine details because the stimuli addressed to each of them are exactly reproduced.

The highest concentration of cones 200 is in the macula 10; cones 200 are mostly concentrated at the fovea 20; only a few are present at the sides of the retina 6.

Defective or damaged rods 100 result in problems seeing in the dark and at night, whereas, defective or damaged cones 200 result in color deficiency.

Because of the structural and functional differences of rods 100 and cones 200, the eye visual field consists of a little central area having high clarity, surrounded by an area having lower clarity in which, however, the sensitivity to light is higher.

The optical rehabilitation treatment according to the present invention takes advantage exactly from the structural and functional differences between rods 100 and cones 200.

In particular, since maculopathy consists in an alteration of the central part of the macula 10, and specifically of the cones 200, while the rods 100 prove to normally function and assure the preservation of both the visual field and the lateral view, the optical rehabilitation treatment according to the present invention, by providing for stimulating the aforesaid rods 100, aims at boosting the lateral view and increasing the overall visual ability of the patient.

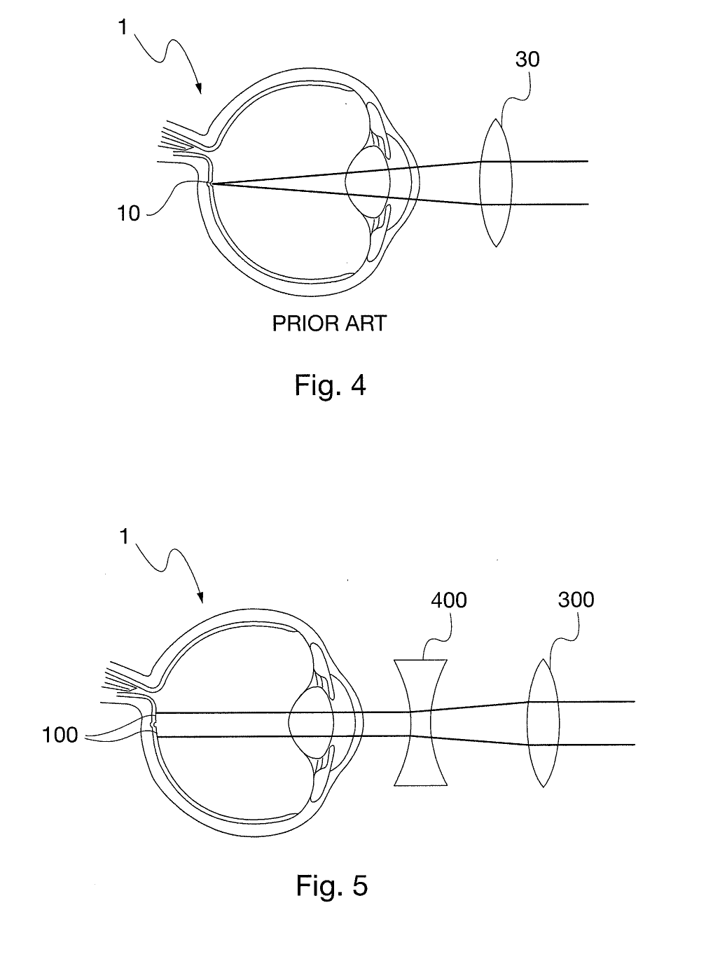

As anticipated and with reference to FIG. 4, prior art treatments try to help patients suffering from maculopathy by providing them with at least one positive, or converging, lens 30; these positive lenses 30 magnify images, thus partially compensating the symptoms due to maculopathy.

Contrary to the above described prior art solution, the optical rehabilitation treatment according to the present invention employs optical means comprising a positive, or converging, lens and a negative, or diverging, lens.

More particularly and with reference to FIG. 5, the aforesaid treatment employs glasses with at least a converging ophthalmic lens 300 in combination with a diverging contact lens 400.

As an alternative, depending on the diagnosed visual pathology, the aforesaid treatment can employ glasses with at least a diverging ophthalmic lens 300 in combination with a converging contact lens 400.

From an operative point of view, after having subjected the patient to an anamnesis of his/her ocular defects, a control of his/her maximum visual acuity is carried out by positioning the patient first at a distance of about 3 meters, and then at a distance of about 1.5 meters, from an acuity chart, so-called octotype.

In case of a nearsighted patient, a control of the glasses for far vision used by the patient himself/herself can be made before checking his/her visual acuity; likewise, in case of a farsighted patient, a control of the glasses for near vision used by the patient himself/herself can be made before checking his/her visual acuity.

Subsequently, after having wetted the patient's cornea with a preservatives-free non-reusable physiological saline solution, at least one diverging contact lens 400 with optical power of −5.00 diopters and, simultaneously, glasses with at least one converging ophthalmic lens 300 with optical power of +5.00 diopters are applied to the patient.

As an alternative, depending on the diagnosed visual pathology, after having wetted the patient's cornea with a preservatives-free non-reusable physiological saline solution, at least one converging contact lens 400 with optical power of +5.00 diopters and, simultaneously, glasses with at least one diverging ophthalmic lens 300 with optical power of −5.00 diopters are applied to the patient.

These initial optical powers are used in order to find the emmetropic photoreceptive area, that is the area without any vision defect.

The above-mentioned optical means 300, 400 are progressively changed by gradually increasing their relevant optical powers up to −100.00 diopters as to the diverging contact lens 400 and up to +100.00 diopters as to the converging ophthalmic lens 300, until a visual improvement is obtained.

As an alternative, depending on the diagnosed visual pathology, the above-mentioned optical means 300, 400 are progressively changed by gradually increasing their relevant optical powers up to +100.00 diopters as to the converging contact lens 400 and up to −100.00 diopters as to the diverging ophthalmic lens 300, until a visual improvement is obtained.

The patient is then let with the optical means 300, 400 on for at least one hour, said optical means 300, 400 consisting of at least one contact lens 400 and glasses with at least one ophthalmic lens 300, the optical power of each of said lenses being that with which the patient recovered the visual function.

After at least one hour has passed, the aforesaid optical means 300, 400 are removed.

The inventor has surprisingly found that, afterwards the rehabilitation treatment carried out at the optician professional's surgery, the recover effect of the visual function remains in time, at least for a period of one month, without requiring that the patient keeps on using the aforesaid neither the aforesaid optical means 300, 400 nor other glasses or lenses; without intending to be bound by the theory, the inventor deems that by using the optical rehabilitation treatment of the invention the patient's brain is conveniently stimulated to receive a well defined peripheral image, it somehow “memorizes” the obtained vision recover and maintains it in time.

After having removed the aforesaid optical means 300, 400, the patient is subjected again to a control of his/her maximum visual acuity and, in case, a myopic or a hyperopic or an astigmatic correction is then applied, to stabilize the optimum visus attained with the rehabilitation treatment.

Finally, in case, the near vision is determined, by making the patient to read a text positioned at a distance from 5 cm to 20 cm.

The treatment duration substantially depends on the intensity of the macular degeneration of each patient; on the basis of the experiments carried out so far, the inventor deems that at least three sessions are needed:
first session: evaluation of the maximum visus attainable for both near and far visions;
second session: after about one week, replication of the treatment according to the invention to confirm the results obtained during first session and, in case, choice of correction glasses for far and/or near vision; and
third session: further replication of the treatment according to the invention and, in case, application of the correction glasses.

Moreover, on the basis of the experiments carried out so far, the inventor deems that the effect of the treatment according to the invention has to be verified after about one month, in order to check if the obtained visual improvement continues; if necessary, said treatment can be repeated.

Of course, the application of the optical means 300, 400 according to the invention can be applied to one or both eyes, taking into account the possible difference in the visual function of the two different eyes; it is worthy to note that particular care must be addressed to the balance of the visual acuities of the two different eyes, in order to avoid causing diplopia.

By way of non limiting example, the results of the experimentation by the inventor are reported hereinafter.

From the month of May 2008 to date, under confidential conditions, the inventor examined 65 patients, 40 of which were without any starting vision defects while 25 of which had starting vision defects (18 of which were nearsighted and 7 of which were farsighted); the treatment to patients with starting vision defects included the correction of the basic vision defect.

The following table summarizes the data relevant to the rehabilitation treatments carried out until now:

| | | | | Recover to |
|---|---|---|---|---|
| | | | Starting visus | visus |
| Number of patients | | 4 | 1/50 | 1/10- 3/10 |
| without starting vision | | 25 | 2/10 | 4/10 |
| defects | | 11 | 3/10- 4/10 | 6/10- 7/10 |
| Number of | Nearsighted | 2 | 1/50 | 1/10- 3/10 |
| patients | patients | 11 | 2/10 | 4/10 |
| with | | 5 | 3/10- 4/10 | 6/10- 7/10 |
| starting | Farsighted | 1 | 1/50 | 1/10- 3/10 |
| vision | patients | 4 | 2/10 | 4/10 |
| defects | | 2 | 3/10- 4/10 | 6/10- 7/10 |

Additionally, two very recent applications have been made, which are reported hereinafter; the first one refers to a choroid retinosis, while the second one refers to a retinitis pigmentosa.

A young man suffering from a choroid retinosis from birth, due to toxoplasmosis, at the right eye, had a starting visus of 2/10; after having been subjected to the treatment of the invention, he recovered to a visus of 8/10, and he maintained in time a recovered visus of 5/10.

The treatment carried out in this case provided the application of a diverging contact lens 400 with optical power of −10.00 diopters and, simultaneously, of glasses with one converging ophthalmic lens 300 with optical power of +9.00 diopters.

It is worthy to note that the treatment according to the invention did not cause any troubles of diplopia.

A patient suffering from a retinitis pigmentosa had a starting visus of 2/10; after having been subjected to the treatment of the invention, he recovered to a visus of 6/10. The treatment carried out in this case provided the application of a converging contact lens 400 with optical power of +5.00 diopters and, simultaneously, of glasses with at least one diverging ophthalmic lens 300 with optical power of −5.00 diopters.

It is worthy to note that the retinitis pigmentosa is a very serious pathology, which damages the rods 100 and leads to blindness; the treatment according to the invention intended to stimulate the fovea 20 and, therefore, the cones 200 rather than the rods 100.

The at least one contact lens 400 according to the invention can have either a spherical or an aspherical geometry; depending on the patient, either soft (with water content from about 35% to about 80%) or rigid gas-permeable contact lenses can be used.

For instance, if the patient does not suffer from astigmatism, soft contact lenses are preferred, while if the patient suffers from astigmatism, semi-rigid to rigid contact lenses are preferred because they have a major amount of fibers.

Also daily-disposable or monthly-disposable contact lenses can be used.

The at least one ophthalmic lens 300 according to the invention is preferably made of crystal.

Even though the inventor devised the optical rehabilitation treatment mainly for the cure of maculopathy, he has already proved its effectiveness as to both choroid retinosis and retinitis pigmentosa; therefore, the inventor deems that said treatment should be satisfactorily used also for the cure of amblyopia, emmetropia with presbyopia as well as of not operable cataracts.

It is important to highlight that the rehabilitation treatment according to the invention must be carried out at an optician professional's surgery, and in no case it can be carried out by the patient himself/herself.

Moreover, it is also important to highlight that the rehabilitation treatment according to the invention can be efficaciously applied to give a vision recovery provided that the rods 100, which are the peripheral photoreceptors, are sound; in case, some cones 200 too are still sound in the macula 10, the vision recovery should be greater.

1. An optical rehabilitation treatment comprising the steps of:
a) subjecting a patient to an anamnesis of his/her ocular defects;
b) controlling the patient's maximum visual acuity by positioning the patient first at a distance of about 3 meters, and then at a distance of about 1.5 meters, from an octotype;
c) wetting the patient's cornea (2) with a preservatives-free non-reusable physiological saline solution
d) applying to the patient at least one contact lens (400) with an initial optical power and, simultaneously, glasses with at least one ophthalmic lens (300) with an initial optical power;
e) progressively changing said optical means (300, 400) by gradually increasing their relevant optical powers until a visual improvement is obtained;
f) letting the patient with said optical means (300, 400) on for at least one hour, the optical power of each of said optical means (300, 400) being that with which the patient recovered the visual function;
g) removing said optical means (300, 400) after at least one hour has passed;
h) subjecting again the patient to a control of his/her maximum visual acuity, to determine the obtained visual improvement.

2. An optical rehabilitation treatment according to claim 1, further comprising the step of:
i) applying a myopic or a hyperopic or an astigmatic correction, to stabilize the optimum visus attained with the rehabilitation treatment.

3. An optical rehabilitation treatment according to claim 1, further comprising the step of:
j) determining the near vision, by making the patient to read a text positioned at a distance from 5 cm to 20 cm.

4. An optical rehabilitation treatment according to claim 1, wherein said step d) comprises:
applying to the patient at least one diverging contact lens (400) with initial optical power of −5.00 diopters and, simultaneously, glasses with at least one converging ophthalmic lens (300) with initial optical power of +5.00 diopters,

5. An optical rehabilitation treatment according to claim 1, wherein said step d) comprises:
applying to the patient at least one converging contact lens (400) with initial optical power of +5.00 diopters and, simultaneously, glasses with at least one diverging ophthalmic lens (300) with initial optical power of −5.00 diopters.

6. An optical rehabilitation treatment according to claim 1, further comprising, before step b), the step of controlling either the glasses for far vision used by the patient in case he/she is a nearsighted patient or the glasses for near vision used by the patient in case he/she is a farsighted patient.

7. An optical rehabilitation treatment according to claim 1, wherein steps a) to h) have to be repeated after about one week to confirm the results obtained during first session and, in case, have to be repeated again to confirm the results obtained during first and second sessions.

8. An optical rehabilitation treatment according to claim 2, wherein steps a) to i) have to be repeated after about one week to confirm the results obtained during first session and to choose correction glasses for far and/or near vision.

9. An optical rehabilitation treatment according to claim 8, wherein steps a) to i) have to be repeated again to confirm the results obtained during first and second sessions and to apply correction glasses.

10. An optical rehabilitation treatment according to claim 1, wherein said step h) has to be repeated after about one month, in order to check if the obtained visual improvement continues.

11. An optical rehabilitation treatment according to claim 1, applicable to one or both eyes of a patient.

12. An optical rehabilitation treatment according to claim 2, further comprising the step of:
j) determining the near vision, by making the patient to read a text positioned at a distance from 5 cm to 20 cm.

10. An optical rehabilitation treatment according to claim 7, wherein said step h) has to be repeated after about one month, in order to check if the obtained visual improvement continues.
